(19) United States
(12) Patent Application Publication
(10) Pub. No.: US 2024/0148419 A1
(43) Pub. Date: May 9, 2024

(54) STERNAL RECONSTRUCTION INTERFACE

(71) Applicant: I. CERAM, Limoges (FR)

(72) Inventors: André KERISIT, Limoges (FR); François BERTIN, Limoges cedex (FR); Jérémy TRICARD, Limoges cedex (FR)

(73) Assignee: I. CERAM, Limoges (FR)

(21) Appl. No.: 18/549,123

(22) PCT Filed: Mar. 11, 2022
(86) PCT No.: PCT/EP2022/056331

§ 371 Date: Sep. 5, 2023

(30) Foreign Application Priority Data
Mar. 12, 2021 (FR) FR2102492

(51) Int. Cl. A61B 17/80; A61B 17/00

(52) U.S. Cl. CPC A61B 17/8076; A61B 2017/00836; A61B 2017/0088; A61B 2017/00889; A61B 2017/00893

(57) ABSTRACT

The invention relates to a sternal reconstruction interface (100) comprising an elongate rectilinear body (101). The sternal reconstruction interface (100) comprises two protruding parts (102A, 102B) extending on either side along the elongate rectilinear body (101), each protruding part (102A, 102B) comprising a bevel, the protruding parts (102A, 102B) extending on one and the same plane along the entire length of the elongate rectilinear body (101), and fixing means (104) comprising at least two through-holes (104).

### FIELD OF THE INVENTION

The present invention relates to the field of implants for sternal closure, in particular the invention relates to a sternal reconstruction interface.

### TECHNICAL BACKGROUND

In the majority of cases, performing heart surgery necessitates a sternotomy to be carried out first. This is a chest incision that serves to provide optimal access to the heart.

In a conventional manner, sternotomy is a procedure wherein a saw is used to cut through the sternum, which is thus split into two hemi-sternums enabling a retractor to be put in place so as to allow access to the heart. At the conclusion of the operation, the retractor is removed and the two hem i-sternums are brought together and maintained in contact in order to enable healing.

The sternotomy closure is generally achieved with sternal wires being wound around the sternum. Although the closure is simple, several complications may occur, e.g.: fracture, sternal nonunion, infection, etc. (Sarr M G, Gott V L, Townsend T R, Mediastinal infection after cardiac surgery, Ann Thorac Surg 1984; 38: 415-423).

In the event of risk of infection or a serious infection occurring, a total sternotomy may be performed with a sternum replacement implant then having to be implanted. Such implants are for example disclosed in the French patent application FR3037803.

Sternal nonunion and infection result in particular from mechanical instability in the sternal region due to respiration, as described by Pierre Zurecki in defending his doctoral thesis entitled “Elaboration du cahier des charges d′un dispositif médical pour fermeture sternale [Development of specifications for a medical device for sternal closure]”, presented on 20 Dec. 2000, at the University of Nancy 1 (France). FIG. 1 from the said thesis (on page 52) thus illustrates the forces that are exerted on either side of the sternotomy during respiration (according to Robicsek et al. J Thorac, Cardiovasc Surg. 2000; 48:1-8):
A: action of a rectus muscle (in the event the second muscle is inactive),
B: tensile forces on either side of the sternotomy due to the muscles, pectorals and elevation of the thoracic cage during inspiration,
C: forces generated by the Vasalva manoeuvre,
D and E: displacements in the antero-posterior direction due to an unbalanced action of the respiratory muscles.

The object of the invention relates to a sternal reconstruction interface that enables enhanced post-operative mechanical stabilisation of the sternum. The object of the present invention is also to limit the risks of sternal nonunion. Another object of the invention is to limit the risks of infections.

### SUMMARY

The invention relates to a sternal reconstruction interface comprising an elongate rectilinear body and comprising two protruding parts, extending on either side along the elongate rectilinear body, each protruding part comprising a bevel, the said protruding parts extending on one and the same plane along the entire length of the elongate rectilinear body, and fixing means comprising at least two through holes.

Advantageously, the sternal reconstruction interface that is the subject matter of the invention enables enhanced post-operative mechanical stabilisation of the sternum. The sternal reconstruction interface makes it possible to limit the risk of sternal nonunion, since the protruding parts at least partially penetrate into the hemi-sternums, thereby integrally securing the sternal reconstruction interface with the hem i-sternums.

Advantageously, the protruding parts run over the entire length of the sternal reconstruction interface, which thus maximises both the zone of contact or penetration between the sternal reconstruction interface and the two hemi-sternums and maintaining of the sternal reconstruction interface in position once it is implanted.

According to one embodiment of the invention, each protruding part is adapted so as to penetrate, at least in part, into the lateral thicknesses of a hem i-sternum when the sternal reconstruction interface is implanted.

Advantageously, the protruding parts can penetrate at least partially into the lateral thicknesses of the hemi-sternums, thereby integrally securing the sternal reconstruction interface with the hem i-sternums.

According to one embodiment of the invention, the sternal reconstruction interface further comprises at least one additional protruding part, preferably two additional protruding parts, extending along the elongate rectilinear body in a plane perpendicular to the first plane, preferably on either side of the elongate rectilinear body, the fixing means comprising at least two through holes on the additional protruding part.

Advantageously, the additional protruding parts make it possible to stabilise the positioning of the sternal reconstruction interface once it is implanted.

According to one embodiment of the invention, the fixing means comprise at least two through holes provided on an additional protruding part.

Advantageously, the through-holes serve to enable easy use of suture wires or ties in order to integrally secure the sternal reconstruction interface to the hem i-sternums and close the sternotomy.

According to one embodiment of the invention, the elongate rectilinear body comprises an upper end and a lower end, with the protruding parts and/or the additional protruding parts comprising a chamfer at the upper and/or lower end.

Advantageously, the chamfers reduce the angularity of the sternal reconstruction interface, which limits the injury risks.

According to one embodiment of the invention, the sternal reconstruction interface comprises of titanium, polyetheretherketone, a ceramic, a porous alumina ceramic, surgical or stainless steel or any other material that is suitable for surgical use.

Advantageously, the sternal reconstruction interface is made of a material having surgical properties.

According to one embodiment of the invention, the sternal reconstruction interface is made of porous alumina ceramic and having a porosity by volume of 45 to 75% and/or a pore size of 100 to 900 μm.

Advantageously, the use of porous alumina ceramic makes possible rapid osseointegration of the sternal reconstruction interface.

According to one embodiment of the invention, the sternal reconstruction interface comprises at least one, preferably two, active ingredient/s.

Advantageously, the use, for example, of at least one active ingredient of such type as an antibiotic makes it possible to reduce the risks of postoperative infection.

According to one embodiment of the invention, the material of the sternal reconstruction interface is porous and loaded with a carrier comprising an active ingredient, preferably the active ingredient is selected from among growth factors, analgesics, antibiotics and anti-cancer drugs, or a combination of two or more active ingredients; the said anti-cancer drug being preferably selected from among:
doxorubicin, cisplatin, methotrexate, ifosfamide, cyclophosphamide, vincristine, dactinomycin, etoposide, denosumab;
and the said antibiotic preferably being selected from among:
beta-lactams, in particular amoxicillin, oxacillin, cloxacillin, ceftriaxone, cefotaxime, ceftazidime, piperacillin, imipenen, ertapenem, ceftaroline, aztreonam, cefepime, cefazolin;
fluoroquinolones, in particular ofloxacin, ciprofloxacin, levofloxacin, oxifloxacin;
aminoglycosides, in particular gentamicin, amikacin;
glycopeptides, in particular vancomycin, teicoplanin;
clindamycin;
clofazimine.

Advantageously, the sternal reconstruction interface may be loaded with at least one active ingredient in order to enhance the in vivo delivery of the said active ingredient.

According to one embodiment of the invention, the material of the sternal reconstruction interface is grafted with at least one antibiotic, at least one aptamer, at least one antibody or combinations thereof, the antibiotic being preferably selected from among:
beta-lactams, in particular amoxicillin, oxacillin, cloxacillin, ceftriaxone, cefotaxime, ceftazidime, piperacillin, imipenen, ertapenem, ceftaroline, aztreonam, cefepime, cefazolin;
clofazimine;
glycopeptides, in particular vancomycin, derivatives thereof, teicoplanin;
lipoglycopeptides, in particular dalbavancin, oritavancin, telavancin, daptomycin.

Advantageously, the sternal reconstruction interface may be grafted with at least one active ingredient in order to enhance the in vivo delivery of the said active ingredient.

According to one embodiment of the invention, the sternal reconstruction interface is used in the closure of a sternotomy.

Advantageously, the sternal reconstruction interface serves to enable enhanced post-operative mechanical stabilisation of the sternum while also limiting the risks of sternal nonunion and infections.

### Definitions

Absent indications to the contrary, the proportions or percentages indicated are by weight.

The term “approximately” followed by a numerical value is used to indicate the said numerical value plus or minus 5%, preferably plus or minus 2.5%, even more preferably plus or minus 1%. According to one embodiment of the invention, when the term “approximately” is followed by a numerical value, this term may be omitted.

The upper end of an implant (or of a prosthesis) refers to the end of the implant which, when the implant is positioned in a human body, is oriented towards the head. The lower end of the implant refers to the end opposite the upper end, that is to say the end oriented towards the feet when the implant is positioned in the human body.

The term “front”, referring to a part or an element of an implant, is used to indicate “anterior” or “ventral” positioning, as opposed to the term “rear”, which is used to indicate “posterior” or “dorsal” positioning.

### DESCRIPTION OF THE FIGURES

FIG. 1 represents the forces that are exerted over two hemi-sternums following a sternotomy owing to respiration.

FIG. 2 represents a perspective view of a sternal reconstruction interface according to one embodiment of the invention.

FIG. 3 represents a front view of a sternal reconstruction interface according to one embodiment of the invention.

FIG. 4 represents a side view of a sternal reconstruction interface according to one embodiment of the invention.

FIG. 5 represents a sternal reconstruction interface according to one embodiment of the invention implanted between two hem i-sternums.

FIG. 6 represents a cross sectional view of a sternal reconstruction interface according to one embodiment of the invention implanted between two hem i-sternums.

### DETAILED DESCRIPTION

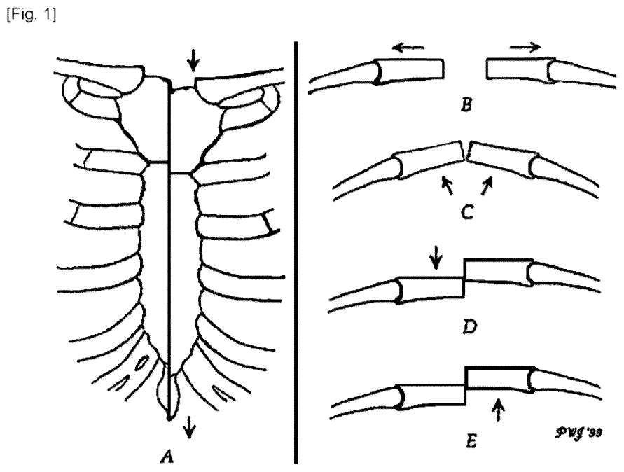

FIG. 1 has previously been described.

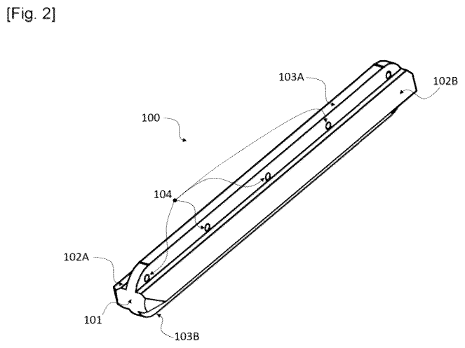

FIG. 2 represents a perspective view of a sternal reconstruction interface 100 according to one embodiment of the invention.

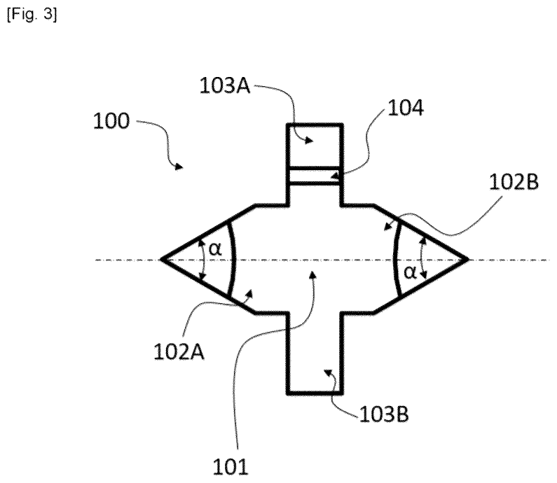

FIG. 3 represents a front view of the sternal reconstruction interface 100.

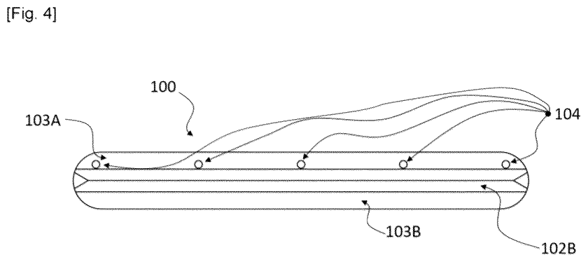

FIG. 4 represents a side view of the sternal reconstruction interface 100.

The sternal reconstruction interface 100 is an implant (or a prosthesis) adapted so as to be positioned between two hemi-sternums following a sternotomy. The sternal reconstruction interface 100 makes it possible to maintain the two hemi-sternums in position so as to be facing one another. The sternal reconstruction interface 100 is also referred to as a sternal closure implant or sternal bar. The sternal reconstruction interface 100 serves as the means for closing the sternum following a sternotomy, while also enabling enhanced postoperative mechanical stabilisation of the sternum. The sternal reconstruction interface 100 also provides the means to limit the risk of nonunion, as well as the risk of infections, for example by being loaded or grafted with one or more antibiotics.

The sternal reconstruction interface 100 comprises an elongate rectilinear body 101 comprising two protruding parts 102A and 102B, extending on either side along the elongate rectilinear body 101, each protruding part 102A and 102B comprising a bevel. The sternal reconstruction interface 100 also comprises fixing means 104.

The elongate rectilinear body 101 extends over a length measuring between a few centimeters and approximately twenty centimeters, this length corresponding substantially to the length of the sternum within which the sternal reconstruction interface 100 is to be inserted. The sternal reconstruction interface 100 may thus exist in various different sizes so as to best fit and be adapted to the sternum that is to be rejoined and stabilised following a sternotomy. The sternal reconstruction interface 100 can be produced in different lengths, which may for example vary between four centimeters and eighteen centimeters, for example in increments of two centimeters.

The fixing means 104 are adapted so as to enable the sternal reconstruction interface 100 to be integrally secured with the two hemi-sternums once it is implanted. The securing may be achieved by means of fasteners, for example wires/filaments, tapes, collars or cords used for sutures. The fixing means 104 may be through holes. The fixing means 104 may be notches. The sternal reconstruction interface 100 may thus comprise one or more fixing means 104. The number of fixing means 104 may depend on the length of the sternal reconstruction interface 100. The fixing means 104 are ideally placed on one front surface of the sternal reconstruction interface 100 in order to be visible and accessible to a surgeon once the sternal reconstruction interface 100 is positioned between the two hem i-sternums.

The sternal reconstruction interface 100 comprises two protruding parts 102A and 102B, extending on either side along the elongate rectilinear body 101, each protruding part 102A and 102B comprising a bevel, preferably a double-bevel.

The protruding parts 102A and 102B run all along the elongate rectilinear body 101, on either side thereof, in order that each may be positioned so as to face the lateral thickness of a corresponding hemi-sternum when the sternal reconstruction interface 100 is positioned between the two hem i-sternums.

According to one embodiment of the invention, both the protruding parts 102A and 102B extend along the same plane. The said plane of extension of the protruding parts 102A and 102B corresponds to a coronal or frontal plane when the sternal reconstruction interface 100 is positioned between the two hemi-sternums. According to an alternative embodiment of the invention, the protruding parts 102A and 102B extend substantially along the same given plane. In other words, each protruding part 102A and 102B defines a plane of extension relative to the elongate rectilinear body 101, these planes forming an angle for example comprised between zero and five degrees.

The protruding parts 102A and 102B each extend over the entire length of the elongate rectilinear body 101. The protruding part 102A and/or 102B may include a chamfered part at its upper and/or lower end. This chamfered part reduces the angularity of the sternal reconstruction interface 100, thereby reducing the risk of injury.

The protruding part 102A and/or 102B comprises a bevel, preferably a double bevel. The bevel, or double bevel, is formed by one or two oblique surfaces that constitute an acute angle. The shape of each protruding part 102A and 102B allows the sternal reconstruction interface 100 to be positioned and maintained in place between the two hemi-sternums, each protruding part 102A and 102B penetrating or being engaged, at least in part, within the lateral thickness of a corresponding hemi-sternum. In other words, each protruding part 102A and 102B bears against and penetrates into the hemi-sternums. The bevel, or double bevel, is not sharp as such. However, the bevel or double bevel is sharp enough such that once a protruding part 102A or 102B is engaged within the lateral thickness of a corresponding hemi-sternum, when the sternal reconstruction interface 100 is positioned between the two hemi-sternums and fixed, the sternal reconstruction interface 100 is maintained in place and is unable to move, particularly in a forward or backward direction.

Preferably, the protruding part 102A and/or 102B comprises a double bevel, which contributes to the sternal reconstruction interface 100 being properly centred once it is implanted, thus preventing any postoperative movement thereof. According to one embodiment of the invention, the angle “a” formed by a tip of a double bevel is between 40° and 80°, preferably between 50° and 70°. According to one embodiment of the invention, the angle “a” formed by a tip of a double bevel is 60°. According to one embodiment of the invention, the bisecting plane of each angle “a” formed by a tip of a double bevel is the plane of extension of the protruding parts 102A and/or 102B. According to one embodiment of the invention, each double bevel is symmetrical, each face of the double bevel forming the same angle “α/2” with the plane of extension of the protruding parts 102A and 102B. In other words, if the angle formed by the tip of a double bevel is 60°, each face of the double bevel forms an angle of 30° with the plane of extension or the bisecting plane.

According to one embodiment of the invention, the sternal reconstruction interface 100 comprises two additional protruding parts 103A and 103B, extending on either side along the elongate rectilinear body 101 in a plane perpendicular to the plane of extension of the protruding parts 102A and 102B.

According to one embodiment of the invention, the sternal reconstruction interface 100 comprises an additional protruding part 103A or 103B, extending along the elongate rectilinear body 101 in a plane perpendicular to the plane of extension of the protruding parts 102A and 102B.

According to one embodiment of the invention, the additional protruding part 103A is placed in front of the sternal reconstruction interface 100 and comprises the fixing means 104. The fixing means 104 thus remain visible and accessible to the surgeon during the operation for putting in place a sternal reconstruction interface 100. The additional protruding part 103A may comprise holes. According to one embodiment of the invention, the part additional protruding part 103A may comprise at least two through holes. Preferably, the holes entirely pass through the thickness of the additional protruding part 103A along a transverse axis, that is to say an axis that is perpendicular to the axis defined by the direction of the elongate rectilinear body 101 and parallel to the plane of extension of the protruding parts 102A and 102B.

The additional protruding parts 103A and/or 103B have flat or substantially flat front and/or rear surfaces.

The fixing means 104 may be distributed along the additional protruding part 103A or 103B. According to one embodiment of the invention, the fixing means 104 are spaced apart successively at equidistance from each other.

The additional protruding part 103B is placed at the rear of the sternal reconstruction interface 100.

The two additional protruding parts 103A and 103B contribute to the stabilising of the sternal reconstruction interface 100 once it has been implanted, by reducing the possibility of the sternal reconstruction interface 100 pivoting around the axis defined by the length of the elongate rectilinear body 101, with each additional protruding part 103A and 103B coming to bear laterally against the respective hemi-sternums.

According to one embodiment of the invention, the sternal reconstruction interface 100 comprises an upper end and a lower end, the elongate rectilinear body 101, the protruding parts 102A and/or 102B, and/or the additional protruding parts 103A and/or 103B comprise one or more chamfers at the upper and/or lower end. Such a chamfer at the upper and/or lower end of the elongate rectilinear body 101, of a protruding part 102A and/or 102B or of an additional protruding part 103A and/or 103B, by averting sharp angles, thus serves to prevent injury after implantation of the sternal reconstruction interface 100.

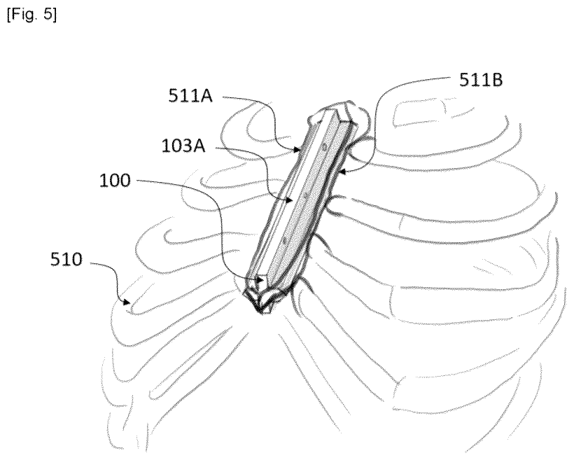

FIG. 5 represents a sternal reconstruction interface 100 according to one embodiment of the invention. The sternal reconstruction interface 100 is implanted between two hemi-sternums 511 A and 511 B of a thoracic cage 510.

The additional protruding part 103A of the sternal reconstruction interface 100 is visibly apparent, with the additional protruding part 103A comprising the fixing means, in this instance three through holes. The sternal reconstruction interface 100 is represented herein as being inserted between the two hemi-sternums 511 A and 511 B, with the fixing means remaining accessible and visible for the surgeon. The surgeon thus is able to easily complete the operation of inserting the sternal reconstruction interface 100, for example, by passing wires through the fixing means in order to integrally secure the sternal reconstruction interface 100 to the two hemi-sternums 511 A and 511 B. According to one embodiment of the invention, the additional protruding part 103A is positioned flush with the surface of the thoracic cage 510 once the sternal reconstruction interface 100 has been implanted. The length of the sternal reconstruction interface 100 is adapted to fit the length of the sternum so as to ensure that the sternal reconstruction interface 100 can be inserted to fit optimally between the two hem i-sternums 511 A and 511 B.

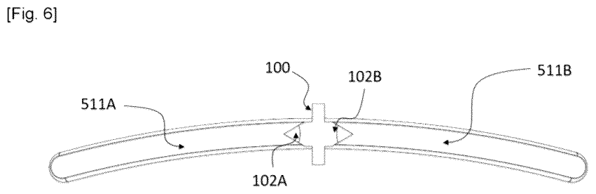

FIG. 6 represents a cross sectional view of a sternal reconstruction interface 100 implanted between the two hem i-sternums 511A and 511B.

As illustrated in FIG. 6, each protruding part 102A and 102B comes to bear against and penetrates into the respective hemi-sternums 511 A and 511 B. The double bevel shaped form of the protruding parts 102A and 102B enables penetration by each protruding part 102A and 102B into the lateral thickness of each respective hemi-sternum 511 A and 511 B. The double bevel shaped form of each protruding part 102A and 102B enables the sternal reconstruction interface 100 to be properly centred in a frontal plane between the two hemi-sternums 511 A and 511 B, and prevents any postoperative movement of the sternal reconstruction interface 100.

Materials

According to one embodiment of the invention, the sternal reconstruction interface 100 is comprised of titanium, “PEEK” (or polyetheretherketone), a ceramic (for example hydroxyapatite), stainless (“inox”) steel or surgical steel or any other material that is suitable for surgical use.

According to one embodiment, the sternal reconstruction interface 100 is made of a material comprising an alumina ceramic. According to one embodiment, the sternal reconstruction interface 100 is made of a material consisting of an alumina ceramic.

The ceramic, that is Al₂O₃ alumina-based, is advantageously porous. While the latter is known per se use may be made of alumina ceramic that is doped with certain other zirconia type materials.

According to one embodiment of the invention, the porosity by volume (open and interconnected) of this ceramic is between 40 and 80%, preferably between 45 and 75%, even more preferably between 60 and 70%, advantageously approximately 65%

According to one embodiment of the invention, the pore size is comprised between 100 and 900 μm, preferably between 200 and 600 μm, advantageously approximately 400 μm.

The porosity/pore size is measured by means of mercury porosimetry. The porosity is defined by the difference between the volume occupied by the pores and the total volume, the total volume being the sum of the volume of the pores and of the alumina. Given that the mass of alumina is defined by its volume and density, by weighing the sample and determining its total volume, it is possible to determine the volume of pores and therefore the (open) porosity as a function of the difference.

The porous alumina allows for secondary osseointegration of the sternal reconstruction interface 100 at approximately 3 months.

According to one embodiment, the mechanical resistance to compression of the sternal reconstruction interface 100 is advantageously between 20 and 60 MPa, advantageously greater than approximately 40 MPa.

Generally, use may be made of any known process for preparing porous alumina; in particular by impregnation with a foam, pre-sintering at a temperature higher than 1200° C., over-impregnation with a barbotine or slip, and sintering at a temperature higher than 1600° C.

According to one embodiment, the porous alumina preparation process comprises the following steps:
(A) provision of a pore-forming material (foam-type material, for example polyurethane foam, used in particular to adjust the porosity and size of pores) and impregnation of the pore-forming material with a suspension of alumina ceramic (alumina barbotine or slip) particles optionally mixed with various organic additives such as binders, plasticisers and dispersants;
(B) oven drying;
(C) low temperature heat treatment (at temperatures preferably lower than 700° C.) in order to eliminate the foam and organic constituents from the suspension; then
(D) sintering at a temperature higher than 1500° C.

The method described in the French patent application FR2823674 may be advantageously used.

In the preferred embodiment, after the implementation of the first two phases as described above (phases A, B), the porous ceramic part is pre-sintered at a temperature higher than 1200° C. (phase C′), whereby it is conferred with the property of superior cohesion. The process continues by immersing the part in another suspension of ceramic particles (phase E). The viscosity of this concentrated suspension is controlled by using various organic auxiliary agents (binders, plasticisers, dispersants), so as to be suitable for bringing about homogeneous impregnation of the pre-sintered porous part. After a further cycle of oven drying (phase B′) and pyrolysis of the organic auxiliary agents in the suspension by means of heat treatment at low temperature, preferably lower than 700° C. (phase C), the ceramic part is finally sintered at a temperature higher than 1600° C. (phase D′).

This process of over-impregnation reinforces the mechanical properties of the sintered ceramic and increases its mechanical strength by a factor of 2, in particular the compressive breaking strength.

Such a ceramic is available with the applicant, under the product reference Ceramil®.

The desired shape and form for the ceramic part may be achieved via machining or by means of a conforming process directly during sintering.

The sternal reconstruction interface 100 may comprise at least one, preferably two, active ingredients. An active ingredient may be selected from among growth factors, analgesics, antibiotics, antibodies, aptamers, anti-cancer drugs, or a combination thereof.

Loading of an Active Ingredient

According to one embodiment, the sternal reconstruction interface 100 is made of a porous material and a carrier comprising an active ingredient is loaded into the sternal reconstruction interface 100. The porous material defines a porous matrix within which an active ingredient may be loaded, possibly by making use of a carrier.

The sternal reconstruction interface 100 may be loaded with one or more active ingredients according to the teaching disclosed in the French patent application FR3027522.

According to one embodiment, the sternal reconstruction interface 100 is a porous ceramic, preferably a porous alumina ceramic, which is loaded.

The carrier is a composition which is used to enable the active ingredient to be retained in the pores. This carrier may be any carrier that is capable of being loaded within the pores, and may in particular be in the form of a gel or in the form of a liquid that may be lyophilised. The carrier is suitable for enabling release of the active ingredient according to a chosen release profile.

The porous matrix of the sternal reconstruction interface 100 may be loaded with the carrier as follows: the carrier is introduced in liquid or gelatinous form into the matrix and thereafter lyophilisation is carried out.

According to one embodiment of the invention, the active ingredient is selected from among growth factors, analgesics, antibiotics and anti-cancer drugs, or a mixture thereof.

According to one embodiment of the invention, the antibiotics are selected from among:
beta-lactams, in particular amoxicillin, oxacillin, cloxacillin, ceftriaxone, cefotaxime, ceftazidime, piperacillin, imipenen, ertapenem, ceftaroline, aztreonam, cefepime, cefazolin;
fluoroquinolones, in particular ofloxacin, ciprofloxacin, levofloxacin, oxifloxacin;
aminoglycosides, in particular gentamicin, amikacin;
glycopeptides, in particular vancomycin, teicoplanin;
clindamycin;
clofazimine.

According to one preferred embodiment of the invention, the antibiotic is gentamicin and/or vancomycin.

The implantation of a sternal reconstruction interface 100 loaded with antibiotics serves two purposes of interest:
protecting the implantation by releasing antibiotics intended for prophylaxis during the time required for healing, in the same manner as antibiotic prophylaxis administered during surgery, but offering the advantage of a massive local concentration that is delivered after closure of the wound and at the time of healing,
delivering locally a large quantity of antibiotics, thereby making it possible to treat a local infection caused by germs which are difficult to sterilise by a systemic route, such as bone infections for example, while also maintaining, for the time being, an antibiotic therapy administered by a systemic route which complies with best practices and standards for the proper use of antibiotics currently in force.

The sternal reconstruction interface 100 provides an effective therapeutic solution for pathologies of the sternum such as osteitis or osteomyelitis, for which the therapeutic alternatives are both limited in number and have a low level of efficacy.

Thanks to their porosity, sternal reconstruction interfaces 100 do not present any physical barrier to release and therefore allow for the release of the totality of the antibiotics they contain.

In vitro, with regard to kinetics, in agitated physiological saline (which does not correspond to physiological conditions in which periprosthetic flows are slower), the product is released in its entirety within approximately three hours.

In vivo, an antibiotic such as gentamicin or vancomycin is released over several hours. Thus, over five implantation procedures performed with sternal reconstruction interfaces 100 loaded with antibiotics, the Redon drainage system showed that the antibiotic was still present twenty-four hours post implantation.

The beneficial purpose of loading a sternal reconstruction interface 100 with an antibiotic (for example gentamicin and/or vancomycin) is to prevent colonisation of the sternal reconstruction interface 100 by bacteria. In fact, the local release of the antibiotic makes it possible to eradicate any bacteria which might persist in the surgical site that was previously infected or that might or could have penetrated the surgical site during the healing phase. The dose of antibiotic released is very high as compared to a local dose that might possibly be prescribed (obtained) during systemic administration, making it possible to optimise the pharmacological criteria for the action of the antibiotics.

Implantation of a sternal reconstruction interface 100 loaded with antibiotics, such as gentamicin or vancomycin, makes it possible to deliver to the core of the infectious site antibiotics that are suitable for treating the pathology, while at the same time enabling early sternal reconstruction that limits the risks inherent in current surgical techniques (pain, diminished strength, sternal instability, etc).

According to one embodiment of the invention, the active ingredient is an anti-cancer agent selected from among agents acting:
against osteosarcomas and spindle cell sarcomas of the bones: doxorubicin, cisplatin, methotrexate, ifosfamide;
against Ewing's sarcomas: doxorubicin, cyclophosphamide, ifosfamide, vincristine, dactinomycin, etoposide;
against giant cell tumours: denosumab;
against soft tissue sarcomas: doxorubicin.

The implantation of a sternal reconstruction interface 100 loaded with anticancer agents serves the beneficial purpose of locally delivering a large quantity of anticancer agents thereby enabling effective localised treatment.

Grafting of an Active Ingredient

According to one embodiment of the invention, an antibiotic is grafted onto the sternal reconstruction interface 100.

According to one preferred embodiment of the invention, the sternal reconstruction interface 100 consists of an alumina ceramic grafted with an antibiotic. The sternal reconstruction interface 100 may comprise of titanium, “PEEK” (or polyetheretherketone), a ceramic (for example hydroxyapatite), stainless (“inox”) steel or surgical steel or any other material that is suitable for surgical use.

The grafting may be performed according to the teaching disclosed in the French patent application FR3074050.

According to one embodiment of the invention, the antibiotic grafted is an antibiotic that has an accessible amine functional group and exhibits an action on the bacterial wall. Antibiotics that exhibit action on the wall are preferred over those that act within the interior of the bacteria.

According to one embodiment of the invention, the antibiotic is selected from among:
beta-lactams, in particular amoxicillin, oxacillin, cloxacillin, ceftriaxone, cefotaxime, ceftazidime, piperacillin, imipenen, ertapenem, ceftaroline, aztreonam, cefepime, cefazolin;
clofazimine;
glycopeptides, in particular vancomycin or derivatives thereof, teicoplanin;
lipoglycopeptides, in particular dalbavancin, oritavancin, telavancin, daptomycin.

In one preferred embodiment of the invention, the selected antibiotic is vancomycin or a derivative, and more preferably vancomycin.

According to the present invention, the antibiotic is present on the surface of the sternal reconstruction interface 100, preferentially on the surface of the alumina ceramic, the surface possibly being the internal surface. According to one embodiment, the antibiotic is grafted via covalent bonds.

According to one embodiment of the invention, the grafting method for grafting an antibiotic onto the alumina ceramic makes it possible to bind, via spacers (or “linkers”), the antibiotic with an amine functional group on the surface of the alumina ceramic.

According to one embodiment of the invention, the sternal reconstruction interface 100 comprises of an alumina ceramic material on which is grafted at least one aptamer and/or at least one antibody that serves to enable the capture of foreign cellular elements circulating in the blood, preferably circulating tumour cells.

The sternal reconstruction interface 100 can be produced according to the teaching disclosed in the French patent application FR3099373.

According to one embodiment of the invention, the alumina ceramic material is grafted with at least one aptamer, at least one antibody, or combinations thereof.

According to one embodiment of the invention, the material of the sternal reconstruction interface is grafted with at least one antibiotic, at least one aptamer, at least one antibody, or combinations thereof.

Plasma-Sprayed Alumina or Hydroxyapatite

The sternal reconstruction interface 100, comprising or constituted of one of the materials listed above, may be covered by a ceramic coating (for example comprising alumina) measuring a few tens to a few hundreds of microns in thickness by using a plasma spraying process.

The sternal reconstruction interface 100, comprising or constituted of one of the materials listed above, may be covered by a hydroxyapatite coating measuring a few tens to a few hundreds of microns in thickness by using a plasma spraying process. The sternal reconstruction interface 100 may thus comprise or be constituted of an alumina ceramic and subsequently be coated with a layer of plasma sprayed hydroxyapatite. The sternal reconstruction interface 100 may comprise or be constituted of titanium, polyetheretherketone, ceramic, porous alumina ceramic, stainless steel or surgical steel, or any other material that is suitable for surgical use, and subsequently be coated with a layer of plasma sprayed hydroxyapatite.

The plasma spraying technique consists in creating an electric arc between an anode and a cathode of a plasma torch. A gaseous mixture, blown through the arc, is heated by the Joule effect and is ionised to form the plasma. The plasma jet is ejected from the nozzle at high speed and high temperatures. The ceramic particles (for example alumina or hydroxyapatite) are then injected into the plasma flow so as to be melted therein, and accelerated towards the surface of the sternal reconstruction interface 100. The droplets, corresponding to the melted particles, then make impact and get flattened on the surface of the sternal reconstruction interface 100 and thus form the desired coating deposit through successive impingement.

Studies have revealed a significant reduction (by a factor of 10) in the adhesion per unit of surface area of bacteria to alumina as compared, for example, to polyethylene. This therefore leads to a reduction in risk of infection during implantation in the human body of the sternal reconstruction interface 100 thus coated with alumina.

Given that hydroxyapatite comprises calcium, coating a sternal reconstruction interface 100 with a layer of plasma sprayed hydroxyapatite thus contributes to enhanced osseointegration of the implanted sternal reconstruction interface 100.

1.-9. (canceled)

10. A sternal reconstruction interface that comprises an elongated rectilinear body comprising:
two protruding parts, extending on either side along the elongated rectilinear body, each protruding part comprising a bevel, said protruding parts extending on a same first plane along the entire length of the elongated rectilinear body,
fixing means comprising at least two through holes.

11. The sternal reconstruction interface according to claim 10, wherein each protruding part is adapted so as to penetrate, at least in part, into the lateral thickness of a hemi-sternum when the sternal reconstruction interface is implanted.

12. The sternal reconstruction interface according to claim 10, further comprising at least one additional protruding part extending along the elongated rectilinear body in a plane perpendicular to the first plane, the fixing means comprising at least two through holes on the additional protruding part.

13. The sternal reconstruction interface according to claim 10, further comprising at least two additional protruding parts extending along the elongated rectilinear body in a plane perpendicular to the first plane on either side of the elongated rectilinear body, the fixing means comprising at least two through holes on the additional protruding part.

14. The sternal reconstruction interface according to claim 10, wherein the elongated rectilinear body comprises an upper end and a lower end, and wherein the protruding parts or the additional protruding parts comprise a chamfer at the upper and/or lower end.

15. The sternal reconstruction interface according to claim 10, wherein the elongated rectilinear body comprises an upper end and a lower end, and wherein the protruding parts and additional protruding parts comprise a chamfer at the upper and/or lower end.

16. The sternal reconstruction interface according to claim 10 comprising titanium, polyetheretherketone, ceramic, porous alumina ceramic, stainless or surgical steel or any other material suitable for surgical use.

17. The sternal reconstruction interface according to claim 16 made of porous alumina ceramic with a porosity by volume of 45 to 75% or a pore size of 100 to 900 μm.

18. The sternal reconstruction interface according to claim 16 made of porous alumina ceramic with a porosity by volume of 45 to 75% and a pore size of 100 to 900 μm.

19. The sternal reconstruction interface according to claim 10 comprising at least one active ingredient.

20. The sternal reconstruction interface according to claim 10 comprising at least two active ingredients.

21. The sternal reconstruction interface according to claim 10, wherein the material of the sternal reconstruction interface is porous and loaded with a carrier comprising at least one active ingredient.

22. The sternal reconstruction interface according to claim 21, wherein the active ingredient is selected from among growth factors, analgesics, antibiotics and anti-cancer drugs, or a combination of two or more active ingredients.

23. The sternal reconstruction interface according to claim 22, wherein the said anti-cancer drug is selected from among doxorubicin, cisplatin, methotrexate, ifosfamide, cyclophosphamide, vincristine, dactinomycin, etoposide, denosumab.

24. The sternal reconstruction interface according to claim 22, wherein the said antibiotic is selected from among beta-lactams; fluoroquinolones; aminoglycosides; glycopeptides; clindamycin; clofazimine.

25. The sternal reconstruction interface according to claim 24, wherein the said antibiotic is selected from among amoxicillin, oxacillin, cloxacillin, ceftriaxone, cefotaxime, ceftazidime, piperacillin, imipenen, ertapenem, ceftaroline, aztreonam, cefepime, cefazolin, ofloxacin, ciprofloxacin, levofloxacin, oxifloxacin, gentamicin, amikacin, vancomycin, teicoplanin.

26. The sternal reconstruction interface according to claim 10, wherein the material of the sternal reconstruction interface is grafted with at least one antibiotic, at least one aptamer, at least one antibody or combinations thereof.

27. Sternal reconstruction interface according to claim 26, wherein the said antibiotic is selected from among beta-lactams; clofazimine; glycopeptides; lipoglycopeptides.

28. Sternal reconstruction interface according to claim 27, wherein the said antibiotic is selected from among amoxicillin, oxacillin, cloxacillin, ceftriaxone, cefotaxime, ceftazidime, piperacillin, imipenen, ertapenem, ceftaroline, aztreonam, cefepime, cefazolin, vancomycin, derivatives thereof, teicoplanin, dalbavancin, oritavancin, telavancin, daptomycin.
